(19) United States
(12) Patent Application Publication
(10) Pub. No.: US 2026/0337697 A1
(43) Pub. Date: Oct. 29, 2026

(54) METHOD AND DEVICE FOR IDENTIFYING TRACHINOTUS ANAK AND DISTINGUISHING BETWEEN WILD POPULATION AND CULTURED POPULATION THEREOF

(71) Applicant: Guangdong Ocean University, Zhanjiang (CN)

(72) Inventors: Yunrong Yan, Zhanjiang (CN); Xiafang Li, Zhanjiang (CN); Zhisen Luo, Zhanjiang (CN); Jiansheng Huang, Zhanjiang (CN); Siwei Zhou, Zhanjiang (CN); Zhao Li, Zhanjiang (CN); Jinxi Wang, Zhanjiang (CN); Jian Luo, Haikou (CN); Chang Wu, Changsha (CN)

(21) Appl. No.: 19/440,685

(22) Filed: Jan. 6, 2026

(30) Foreign Application Priority Data
Apr. 25, 2025 (CN) 202510525782.9

(51) Int. Cl. C12Q 1/6888; G16B 10/00; G16B 30/10

(52) U.S. Cl. CPC C12Q 1/6888; G16B 10/00; G16B 30/10; C12Q 2600/156

(57) ABSTRACT

A method for identifying Trachinotus anak and distinguishing between wild population and cultured population thereof includes: collecting a golden pompano sample to be identified; performing preliminary morphological identification, morphometric measurement, DR imaging, and DNA extraction on the sample; preliminarily determining a species of the sample based on morphological characteristics by using a dichotomous key; amplifying the DNA with specified primers; aligning a sequence of a amplification product with COI sequences downloaded from an NCBI database to construct a phylogenetic tree, and determining the species of the sample at a molecular level; and finally determining the species of the sample by integrating morphological and molecular biological results. The method establishes a discriminant function equation through stepwise discriminant analysis performed on 22 standardized morphometric data from 108 Trachinotus anak specimens. This allows for precise, rapid, and high-throughput identification of Trachinotus anak and discrimination between its wild and cultured populations.

### CROSS-REFERENCE TO RELATED APPLICATION

This application claims priority to Chinese Patent Application No. 202510525782.9, filed on Apr. 25, 2025, which is herein incorporated by reference in its entirety.

### TECHNICAL FIELD

The disclosure relates to the technical field of morphological and molecular marker identification, and more particularly to a method and a device for identifying Trachinotus anak and distinguishing between wild population and cultured population thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 25080THXT-USP1-US-2025-0076-SL.xml. The XML file is 70,343 bytes; is created on Dec. 26, 2025; and is being submitted electronically via patent center.

### BACKGROUND

Golden pompano belongs to the class Actinopterygii, order Perciformes, family Carangidae and genus Trachinotus, and is a major marine aquaculture fish species in Asia. The name “golden pompano” is used to refer to different species, including Trachinotus ovatus, Trachinotus mookalee, Trachinotus anak, and Trachinotus blochii. The first three are collectively known as short-finned golden pompano, while the last is known as long-finned golden pompano. The taxonomy of these species has long been unclear, which is detrimental to the conservation of golden pompano germplasm resources and hinders the sustainable development of aquaculture industry. In addition, it is difficult to distinguish between wild population and cultured population, leading to phenomena such as cultured fish being misrepresented as wild fish, which not only infringes upon the legitimate rights and interests of consumers but also disrupts market competition order.

At present, there is a lack of relevant research on the taxonomy of golden pompano. Therefore, it is of great significance for the conservation of wild population germplasm resources of Trachinotus anak and the sustainable development of the aquaculture industry to establish an accurate, simple, and rapid method for identifying Trachinotus anak and distinguishing between wild population and cultured population thereof.

### SUMMARY

A purpose of the disclosure is to overcome at least one deficiency in the related art by providing a method for identifying Trachinotus anak and distinguishing between wild population and cultured population thereof.

To achieve the above purpose, the disclosure adopts the following technical solution.

Specifically, the disclosure provides a method for identifying Trachinotus anak and distinguishing between wild population and cultured population thereof, which includes:
obtaining a golden pompano sample to be identified;
performing morphometric measurement on the golden pompano sample to be identified to obtain morphometric data of the golden pompano sample to be identified, and performing digital radiography (DR) imaging on the golden pompano sample to be identified to obtain a DR imaging result of the golden pompano sample to be identified;
determining, based on the morphometric data of the golden pompano sample to be identified, the DR imaging result of the golden pompano sample to be identified, and a pre-established dichotomous key, whether the golden pompano sample to be identified is Trachinotus anak, thereby to obtain a first determination result;
performing deoxyribonucleic acid (DNA) extraction on the golden pompano sample to be identified to obtain DNA of the golden pompano sample to be identified;
performing, using the DNA of the golden pompano sample to be identified as a DNA template, polymerase chain reaction (PCR) amplification with primers to obtain an amplification product of the golden pompano sample to be identified, and purifying and sequencing the amplification product to obtain a sequencing result of the golden pompano sample to be identified;
aligning the sequencing result of the golden pompano sample to be identified with cytochrome c oxidase subunit I (COI) sequences downloaded from a national center for biotechnology information (NCBI) database to construct a phylogenetic tree, and determining, based on the phylogenetic tree, whether the golden pompano sample is Trachinotus anak, thereby to obtain a second determination result;
in response to both the first determination result and the second determination result indicating that the golden pompano sample is Trachinotus anak, identifying the golden pompano sample to be identified as Trachinotus anak; and
standardizing the morphometric data to obtain standardized morphometric data of the golden pompano sample to be identified, substituting the standardized morphometric data into a discriminant function equation to obtain a calculation result, and determining, based on the calculation result of the discriminant function equation, a category of the golden pompano sample to be identified within Trachinotus anak, where the category includes the cultured population and the wild population.

In an embodiment, the method for identifying Trachinotus anak and distinguishing between the wild population and the cultured population thereof further includes: generating, based on a category determination result, a traceability code for the golden pompano sample to be identified, printing a corresponding “Wild-Certified” or “Cultured-Certified” label to the golden pompano sample to be identified to obtain a resulting Trachinotus anak, and selling, based on the corresponding label, the resulting Trachinotus anak for sustainable development. In another embodiment, the method further includes: finely targeting, in response to the golden pompano sample printed with the “Wild-Certified” label, the wild population to protect germplasm resources in the wild population. Specifically, in an application of fishery resource protection and scientific management, based on the traceability code for Trachinotus anak, fraudulent practice of selling cultured Trachinotus anak as wild Trachinotus anak is effectively identified and penalized, thereby protecting wild resources from overfishing and commercial fraud, and fishing and trade flows of wild Trachinotus anak are accurately monitoring by a relevant department, thereby assisting to formulate scientific management measures such as catch quotas and closed seasons to ensure sustainable use of fishery resource.

In an embodiment, specifically, the morphometric data of the golden pompano sample to be identified includes:
a fork length FL, a distance Sn-D1O from a snout tip to a first-dorsal-fin origin, a distance Sn-D2O from the snout tip to a second-dorsal-fin origin, a distance Sn-P2O from the snout tip to a pelvic-fin origin, a distance Sn-A2O from the snout tip to an anal-fin origin, a distance D1O-P2O from the first-dorsal-fin origin to the pelvic-fin origin, a distance D1O-A2O from the first-dorsal-fin origin to the anal-fin origin, a distance D2O-P2O from the second-dorsal-fin origin to the pelvic-fin origin, a distance D2O-A2O from the second-dorsal-fin origin to the anal-fin origin, a midbody depth MBD, a half body depth at a pectoral-fin origin P1-D2I, a second-dorsal-fin length HD2L, an anal-fin length HAL, a pectoral-fin length PIL, a pelvic-fin length P2L, a second-dorsal-fin base length LDB, an anal-fin base length LAB, a head length HL, a post-orbital head length PoHL, an eye diameter ED, a snout length SnL, an upper-jaw length UjL, and a maxilla depth MaD.

In an embodiment, specifically, wherein the pre-established dichotomous key is as follows:
1(2) Body oval; body silvery-white with multiple oval dark spots on sides; dorsal and anal fins white with black tips; caudal fin black with a white margin only . . . Trachinotus ovatus (eastern Atlantic)
2(1) Body rhomboidal; body golden-yellow with a grayish-yellow dorsal area, or body silvery-white with a grayish-green dorsal area; no spots on sides; dorsal and anal fins yellow without black tips; caudal fin yellow.
3(4) The first interneural bone oval bead-shaped; anterior rays of second dorsal fin distinctly elongated, 1.5 to 2.0 times the head length, or 35% to 60% of the fork length for individuals of 100 millimeters (mm) to 400 mm . . . Trachinotus blochii (Indo-Pacific)
4(3) The first interneural bone inverted L-shaped; anterior rays of both second dorsal and anal fins relatively short; the second-dorsal-fin length 24% to 30% of the fork length.
5(6) Ventral ribs not enlarged; no increase in bone density observed in preorbital and nasal bones; supraoccipital bone widened, appearing as an elongated elliptical cylinder in individuals with the fork length exceeding 200 mm, more pronounced in individuals with the fork length exceeding 300 mm . . . Trachinotus mookalee (Indo-Pacific)
6(5) Supraoccipital bone thin and blade-shaped; second or enlarged third ventral ribs enlarged; increased bone density observed in preorbital and nasal bones in individuals with the fork length exceeding 300 mm . . . Trachinotus anak (Western Pacific)

In an embodiment, specifically, the primers used for the PCR amplification include:
a FishF1 primer, where the sequence of the FishF1 primer is as shown in SEQ ID NO: 50: 5′-TCAACCAACCACAAAGACATTGGCAC-3′; and
a FishR1 primer, where the sequence of the FishR1 primer is as shown in SEQ ID NO: 51: 3′-TAGACTTCTGGGTGGCCAAACAATCA-5′;
where a system for the PCR amplification comprises: 2 microliters (μL) of the DNA template, 12.5 μL of TaqPCR Mix, 6.5 μL of double-distilled water (ddH₂O), 2 μL of magnesium chloride (MgCl₂), 1 μL of the FishF1 primer, and 1 μL of the FishR1 primer; and
where procedures for the PCR amplification include: pre-denaturation at 95 °C for 3 minutes; 35 cycles of denaturation at 92 °C for 45 seconds, annealing at 50 °C for 45 seconds, and extension at 72 °C for 1 minute; and finally extension at 72 °C for 10 minutes.

In an embodiment, specifically, the aligning the sequencing result of the golden pompano sample to be identified with COI sequences downloaded from an NCBI database to construct a phylogenetic tree, and determining, based on the phylogenetic tree, whether the golden pompano sample to be identified is Trachinotus anak, thereby to obtain a second determination result includes:
aligning a sequence of the amplification product in the sequencing result of the golden pompano sample to be identified with the COI sequences downloaded from the NCBI database to construct the phylogenetic tree, where the COI sequences downloaded from the NCBI database are as shown in SEQ ID NO: 5 to SEQ ID NO: 49, SEQ ID NO: 5 to SEQ ID NO: 47 are the COI sequences from the genus Trachinotus, and SEQ ID NO: 48 and SEQ ID NO: 49 are the outgroup sequences required for constructing the phylogenetic tree; and in response to the sequence of the amplification product clustering into a clade with a COI sequence corresponding to Trachinotus anak in the phylogenetic tree, identifying, by molecular marker identification, the golden pompano sample to be identified as Trachinotus anak.

In an embodiment, specifically, the standardizing the morphometric data of the golden pompano sample to be identified to obtain standardized morphometric data includes:
dividing each of the post-orbital head length PoHL, the eye diameter ED, the snout length SnL, the upper-jaw length UjL, and the maxilla depth MaD by the head length HL, and dividing each of remaining morphometric data of the morphometric data by the fork length FL.

In an embodiment, specifically, the discriminant function equation is constructed based on a stepwise discriminant method, including:
constructing a statistical model from the standardized morphometric data by using a Wilks' lambda method, using an F-distribution value as a criterion, introducing a variable into the statistical model when a F-value of the variable is greater than 3.84, and removing a variable when a F-value of the variable is less than 2.71, thereby ultimately screening out the following five standardized morphometric data from the standardized morphometric data: the distance from the snout tip to the pelvic-fin origin divided by the fork length, denoted as X1; the half body depth at the pectoral-fin origin divided by the fork length, denoted as X2; the anal-fin base length divided by the fork length, denoted as X3; the post-orbital head length divided by the head length, denoted as X4; and the eye diameter divided by the head length, denoted as X5; and
where the discriminant function equation includes a discriminant equation for the wild population and a discriminant equation for the cultured population;
where the discriminant equation for the wild population is as follows:

F1=3504.230*X1−650.078*X2+2317.691*X3+1635.079*X4+1324.686*X5−1467.609;
where the discriminant equation for the cultured population is as follows:

F2=3428.068*X1−596.227*X2+2251.891*X3+1661.023*X4+1373.527*X5−1462.008.

In an embodiment, specifically, the determining, based on the calculation result of the discriminant function equation, a category of the golden pompano sample to be identified within Trachinotus anak includes:
calculating values of F1 and F2 for the golden pompano sample to be identified, and assigning the category of the golden pompano sample to be identified within Trachinotus anak as a category of a corresponding discriminant equation associated with a higher value between the values of F1 and F2.

The disclosure further provides a device for identifying Trachinotus anak and distinguishing between wild population and cultured population thereof, which includes:
a sample acquisition module, configured to obtain a golden pompano sample to be identified;
a data acquisition module, configured to perform morphometric measurement on the golden pompano sample to be identified to obtain morphometric data of the golden pompano sample to be identified, and perform DR imaging on the golden pompano sample to be identified to obtain a DR imaging result of the golden pompano sample to be identified;
a first determination module, configured to determine, based on the morphometric data of the golden pompano sample to be identified, the DR imaging result of the golden pompano sample to be identified, and a pre-established dichotomous key, whether the golden pompano sample to be identified is Trachinotus anak, thereby to obtain a first determination result;
a DNA extraction module, configured to perform DNA extraction on the golden pompano sample to be identified to obtain DNA of the golden pompano sample to be identified;
a PCR amplification module, configured to perform, using the DNA of the golden pompano sample to be identified as a DNA template, PCR amplification with primers to obtain an amplification product of the golden pompano sample to be identified, and purify and sequence the amplification product to obtain a sequencing result of the golden pompano sample to be identified;
a second determination module, configured to align the sequencing result of the golden pompano sample to be identified with COI sequences downloaded from an NCBI database to construct a phylogenetic tree, and determine, based on the phylogenetic tree, whether the golden pompano sample to be identified is Trachinotus anak, thereby to obtain a second determination result;
a species determination module, configured to identify the golden pompano sample to be identified as Trachinotus anak in response to both the first determination result and the second determination result indicating that the golden pompano sample to be identified is Trachinotus anak; and
a category determination module, configured to standardize the morphometric data to obtain standardized morphometric data of the golden pompano sample to be identified, substitute the standardized morphometric data into a discriminant function equation to obtain a calculation result, and determine, based on the calculation result of the discriminant function equation, a category of the golden pompano sample to be identified within Trachinotus anak, where the category includes the cultured population and the wild population.

The method for identifying Trachinotus anak and distinguishing between wild population and cultured population thereof of the disclosure provides the COI sequences for identifying Trachinotus anak and establishes the discriminant function equation for differentiating its wild population and cultured population. The method provided by the disclosure is economical, simple, and highly reproducible, enabling high-throughput identification of Trachinotus anak. It allows for the determination of population origin based on morphometric data without harming Trachinotus anak, which is advantageous for the conservation of wild population germplasm resources of Trachinotus anak and for the sustainable development of aquaculture industry.

### BRIEF DESCRIPTION OF DRAWINGS

By providing a detailed explanation of embodiments shown in attached drawings, the above and other features of the disclosure will become more apparent. Same reference signs in the attached drawings represent same or similar elements. Obviously, attached drawings described below are only some embodiments of the disclosure. For those skilled in the art, other attached drawings can be obtained based on these attached drawings without creative efforts.

FIG. 1 illustrates a schematic flowchart of a method for identifying Trachinotus anak and distinguishing between wild population and cultured population thereof according to disclosure.

FIG. 2A and FIG. 2B illustrate schematic diagrams of morphometric measurement of golden pompano according to the disclosure.

FIG. 3 illustrates a schematic diagram of DR imaging results of Trachinotus anak, Trachinotus mookalee, and Trachinotus blochii.

FIG. 4 illustrates a schematic diagram of a phylogenetic tree of genus Trachinotus.

### DETAILED DESCRIPTION OF EMBODIMENTS

The following will provide a clear and complete description of the concept, specific structure, and technical effects of the disclosure in conjunction with embodiments and attached drawings, in order to fully understand the purpose, scheme, and technical effects of the disclosure. It should be noted that the embodiments and features in the embodiments of the disclosure can be combined with each other without conflict. Same reference signs used in the attached drawings indicate same or similar parts.

The disclosure collects golden pompano samples of varying sizes from cultured and wild populations. Skeletal analysis reveals that in fish with a fork length exceeding 300 mm, preorbital and nasal bones exhibits increased bone density, and second and third ventral ribs are enlarged and also show increased bone density. Compared with type specimens, these samples are identified as Trachinotus anak. A phylogenetic tree constructed based on COI sequences shows that all sequences cluster with sequences identified as Trachinotus anak. Based on the above results, the commonly encountered golden pompano is Trachinotus anak, not Trachinotus ovatus. The true Trachinotus ovatus is found only in the eastern Atlantic Ocean. Traditionally, the presence of tongue teeth has been used to distinguish Trachinotus anak (toothless) from Trachinotus mookalee (toothed). However, the disclosure finds that most Trachinotus anak samples also possess tongue teeth, indicating that this characteristic is not a reliable distinguishing feature.

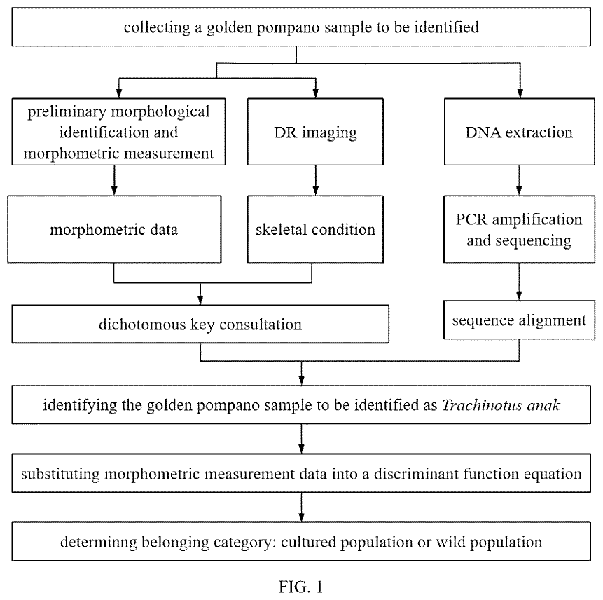

Based on the above analysis, as shown in FIG. 1, an embodiment 1 of the disclosure provides a method for identifying Trachinotus anak and distinguishing between wild population and cultured population thereof, and specific operations of this method are as follows.

A golden pompano sample to be identified is obtained.

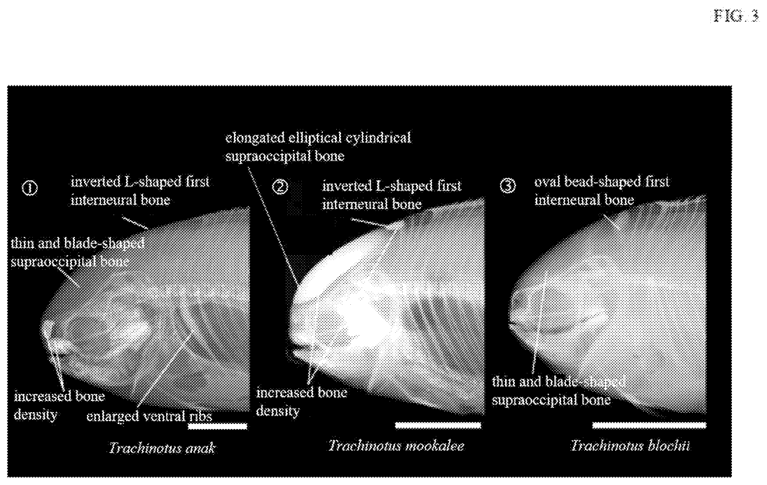

Morphometric measurement is performed on the golden pompano sample to be identified to obtain morphometric data of the golden pompano sample to be identified, and DR imaging is performed on the golden pompano sample to be identified to obtain a DR imaging result of the golden pompano sample to be identified (referring to FIG. 3).

Based on the morphometric data, the DR imaging result, and a pre-established dichotomous key (where the retrieval and comparison method may be manual comparison or may involve establishing a relevant neural network model, and the neural network model performs feature extraction and classification on the dichotomous key to automatically recognize the input morphometric data and the DR imaging result to obtain a recognition result, and determine whether the golden pompano sample to be identified is Trachinotus anak according to a dichotomous key category to which the recognition result belongs), a first determination result is obtained by determining whether the golden pompano sample to be identified is Trachinotus anak (when the manual comparison is used, a manually input comparison result is directly acquired; when relevant neural network model is used to perform intelligent recognition, the recognition result is directly acquired and may be verified and corrected manually).

DNA extraction is performed on the golden pompano sample to be identified to obtain DNA of the golden pompano sample to be identified.

By using the DNA of the golden pompano sample to be identified as a DNA template, PCR amplification is performed with primers to obtain an amplification product of the golden pompano sample to be identified, and the amplification product is purified and sequenced to obtain a sequencing result of the golden pompano sample to be identified.

The sequencing result of the golden pompano sample to be identified is aligned with COI sequences downloaded from an NCBI database to construct a phylogenetic tree. Based on the phylogenetic tree, it is determined whether the golden pompano sample to be identified is Trachinotus anak, thereby to obtain a second determination result.

The COI sequences downloaded from the NCBI database are as shown in SEQ ID NO: 5 to SEQ ID NO: 49. Specifically, SEQ ID NO: 5 to SEQ ID NO: 47 are the COI sequences from the genus Trachinotus, and SEQ ID NO: 48 and SEQ ID NO: 49 are the outgroup sequences required for constructing the phylogenetic tree.

When both the first determination result and the second determination result indicates that the golden pompano sample to be identified is Trachinotus anak, the golden pompano sample to be identified is identified as Trachinotus anak.

The morphometric data is standardized to obtain standardized morphometric data of the golden pompano sample to be identified, and the standardized morphometric data is substitute into a discriminant function equation to obtain a calculation result. Based on the calculation result of the discriminant function equation, a category of the golden pompano sample to be identified within Trachinotus anak is determined, where the category includes the cultured population and the wild population (i.e., the category is the cultured population or the wild population).

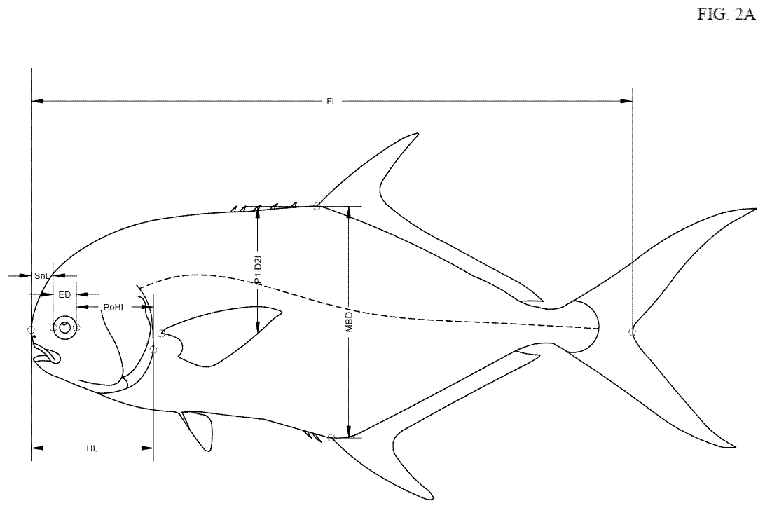

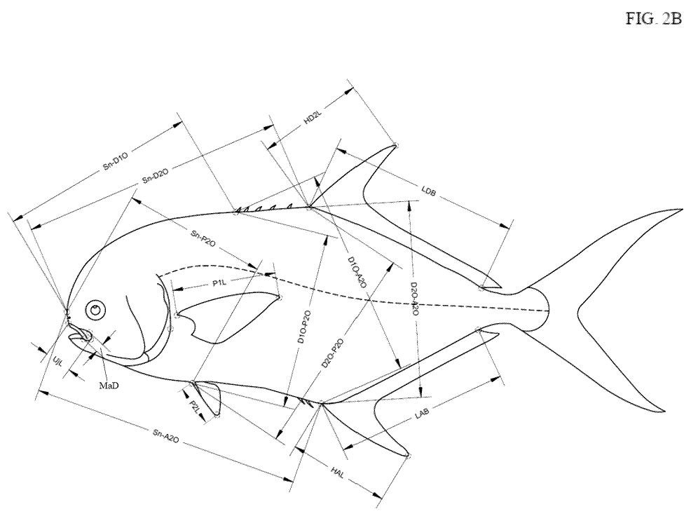

As shown in FIG. 2A and FIG. 2B, in a specific embodiment of the disclosure, the morphometric data of the golden pompano sample to be identified includes: a fork length FL, a distance Sn-D1O from a snout tip to a first-dorsal-fin origin, a distance Sn-D2O from the snout tip to a second-dorsal-fin origin, a distance Sn-P2O from the snout tip to a pelvic-fin origin, a distance Sn-A2O from the snout tip to an anal-fin origin, a distance D1O-P2O from the first-dorsal-fin origin to the pelvic-fin origin, a distance D1O-A2O from the first-dorsal-fin origin to the anal-fin origin, a distance D2O-P2O from the second-dorsal-fin origin to the pelvic-fin origin, a distance D2O-A2O from the second-dorsal-fin origin to the anal-fin origin, a midbody depth MBD, a half body depth at a pectoral-fin origin P1-D2I, a second-dorsal-fin length HD2L, an anal-fin length HAL, a pectoral-fin length PIL, a pelvic-fin length P2L, a second-dorsal-fin base length LDB, an anal-fin base length LAB, a head length HL, a post-orbital head length PoHL, an eye diameter ED, a snout length SnL, an upper-jaw length UjL, and a maxilla depth MaD.

In a specific embodiment of the disclosure, the pre-established dichotomous key is as follows.
1(2) Body oval, body silvery-white with multiple oval dark spots on sides; dorsal and anal fins white with black tips; caudal fin black with a white margin only . . . Trachinotus ovatus (eastern Atlantic)

Herein, the pre-established dichotomous key is in a continuous format. A number at a beginning of a line indicates an item number identifying morphological characters of a current item. A number in parentheses indicates a target item to jump to when the morphological characters of the current item are not satisfied.

In this item, “1” is an item number. When morphological characters of the item 1 are met, a species of the golden pompano sample to be identified is determined as Trachinotus ovatus. When the morphological characters of item 1 are not met, a process proceeds to item 2.
2(1) Body rhomboidal; body golden-yellow with a grayish-yellow dorsal area, or body silvery-white with a grayish-green dorsal area; no spots on sides; dorsal and anal fins yellow without black tips; caudal fin yellow.

Herein, “2” is an item number. When morphological characters of the item 2 are met, the process proceeds to item 3 to check remaining morphological characters.

When the morphological characters of the item 2 are not met, the process proceeds to the item 1.
3(4) First interneural bone oval bead-shaped; anterior rays of second dorsal fin distinctly elongated, 1.5 to 2.0 times the head length, or 35% to 60% of the fork length for individuals of 100 mm to 400 mm . . . Trachinotus blochii (Indo-Pacific)

Herein, “3” is an item number. When morphological characters of the item 3 are met, the species of the golden pompano sample to be identified is determined as Trachinotus blochii. When the morphological characters of the item 3 are not met, the process proceeds to item 4.
4(3) First interneural bone inverted L-shaped; anterior rays of both second dorsal and anal fins relatively short; the second-dorsal-fin length 24% to 30% of the fork length.

Herein, “4” is an item number. When morphological characters of the item 4 are met, the process proceeds to item 5 to check remaining morphological characters. When the morphological characters of item 4 are not met, the process proceeds to the item 3.
5(6) Ventral ribs not enlarged; no increase in bone density observed in preorbital and nasal bones; supraoccipital bone widened, appearing as an elongated elliptical cylinder in individuals with the fork length exceeding 200 mm, more pronounced in individuals with the fork length exceeding 300 mm . . . Trachinotus mookalee (Indo-Pacific)

Herein, “5” is an item number. When morphological characters of the item 5 are met, the species of the golden pompano sample to be identified is determined as Trachinotus mookalee. When the morphological characters of the item 5 are not met, the process proceeds to item 6.
6(5) Supraoccipital bone thin and blade-shaped; second or enlarged third ventral ribs enlarged; increased bone density observed in preorbital and nasal bones in individuals with the fork length exceeding 300 mm . . . Trachinotus anak (Western Pacific)

Herein, “6” is an item number. When morphological characters of the item 6 are met, the species of the golden pompano sample to be identified is determined as Trachinotus anak. When the morphological characters of the item 6 are not met, the process proceeds to item 5.

In a specific embodiment of the disclosure, the primers used for the PCR amplification include a FishF1 primer and a FishR1 primer.

Specifically, the sequence of the FishF1 primer is as shown in SEQ ID NO: 50: 5′-TCAACCAACCACAAAGACATTGGCAC-3′.

The sequence of the FishR1 primer is as shown in SEQ ID NO: 51: 3′-TAGACTTCTGGGTGGCCAAACAATCA-5′.

A system for the PCR amplification includes: 2 μL of the DNA template, 12.5 μL of TaqPCR Mix, 6.5 μL of ddH₂O, 2 μL of MgCl₂, 1 μL of the FishF1 primer, and 1 μL of the FishR1 primer. Specifically, the TaqPCR Mix contains Taq DNA polymerase, dNTPs, MgCl₂, PCR buffer, PCR enhancer, stabilizers, and a blue tracking dye and is produced by BBI Life Sciences. The ddH₂O is treated with diethyl pyrocarbonate (DEPC), free from impurities such as ribonuclease (RNase) and deoxyribonuclease (DNase), and is produced by Sangon Biotech. The MgCl₂ 25 is a millimoles per liter (mM) MgCl₂ sterile aqueous solution and is produced by BBI Life Sciences.

Procedures for the PCR amplification include: pre-denaturation at 95 °C for 3 minutes; 35 cycles of denaturation at 92 °C for 45 seconds, annealing at 50 °C for 45 seconds, and extension at 72 °C for 1 minute; and finally extension at 72 °C for 10 minutes.

In a specific embodiment, the step of aligning the sequencing result of the golden pompano sample to be identified with the COI sequences downloaded from the NCBI database to construct the phylogenetic tree, and determining, based on the phylogenetic tree, whether the golden pompano sample to be identified is Trachinotus anak, thereby to obtain a second determination result includes the following operations.

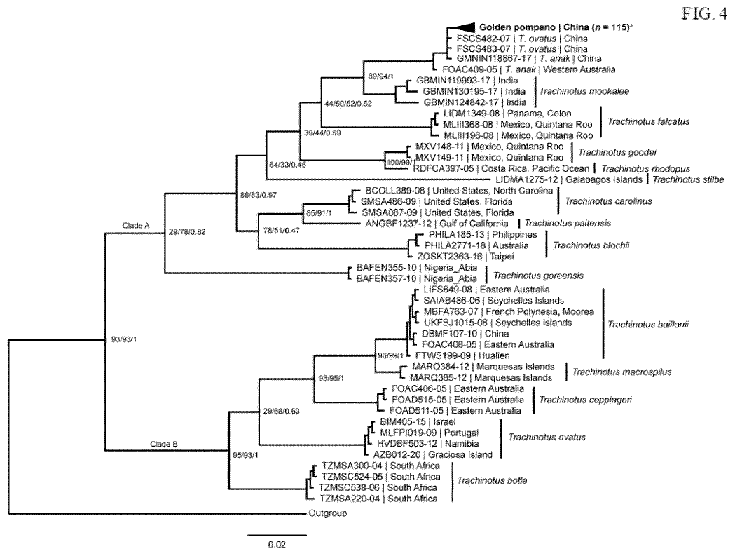

A sequence of the amplification product in the sequencing result of the golden pompano sample to be identified is aligned with the COI sequences downloaded from the NCBI database to construct the phylogenetic tree (referring to FIG. 4). When the sequence of the amplification product clusters into a clade with a COI sequence corresponding to Trachinotus anak in the phylogenetic tree, it is identified by molecular marker identification that the golden pompano sample to be identified is Trachinotus anak.

The COI sequences downloaded from the NCBI database are as shown in SEQ ID NO: 5 to SEQ ID NO: 49. Specifically, SEQ ID NO: 5 to SEQ ID NO: 47 are the COI sequences from the genus Trachinotus, and SEQ ID NO: 48 and SEQ ID NO: 49 are the outgroup sequences required for constructing the phylogenetic tree.

In a specific embodiment, the morphometric data of the golden pompano sample to be identified is standardized as follows.

Each of the post-orbital head length PoHL, the eye diameter ED, the snout length SnL, the upper-jaw length UjL, and the maxilla depth MaD is divided by the head length HL, and each of remaining morphometric data of the morphometric data is divided by the fork length FL. Specifically, the remaining morphometric data includes the distance Sn-D1O from the snout tip to the first-dorsal-fin origin, the distance Sn-D2O from the snout tip to the second-dorsal-fin origin, the distance Sn-P2O from the snout tip to the pelvic-fin origin, the distance Sn-A2O from the snout tip to the anal-fin origin, the distance D1O-P2O from the first-dorsal-fin origin to the pelvic-fin origin, the distance D1O-A2O from the first-dorsal-fin origin to the anal-fin origin, the distance D2O-P2O from the second-dorsal-fin origin to the pelvic-fin origin, the distance D2O-A2O from the second-dorsal-fin origin to the anal-fin origin, the midbody depth MBD, the half body depth at the pectoral-fin origin P1-D2I, the second-dorsal-fin length HD2L, the anal-fin length HAL, the pectoral-fin length PIL, the pelvic-fin length P2L, the second-dorsal-fin base length LDB, the anal-fin base length LAB.

In a specific embodiment, the discriminant function equation is constructed based on a stepwise discriminant method, which includes the following operations.

A statistical model is constructed from the standardized morphometric data by using a Wilk's lambda method, and an F-distribution value is used as a criterion. A variable is introduced into the statistical model when a F-value of the variable is greater than 3.84, and a variable is removed when a F-value of the variable is less than 2.71. Through stepwise variable introduction, 22 standardized morphometric data are gradually introduced over 5 operational steps. Then, variables (i.e., standardized morphometric data) with small contribution rates to the statistical model are gradually eliminated according to their contribution rates. Ultimately, five standardized morphometric data are selected from the standardized morphometric data: the distance from the snout tip to the pelvic-fin origin divided by the fork length, denoted as X1; the half body depth at the pectoral-fin origin divided by the fork length, denoted as X2; the anal-fin base length divided by the fork length, denoted as X3; the post-orbital head length divided by the head length, denoted as X4; and the eye diameter divided by the head length, denoted as X5.

The discriminant function equation includes a discriminant equation for the wild population and a discriminant equation for the cultured population.

Specifically, the discriminant equation for the wild population is as follows:

F1=3504.230*X1−650.078*X2+2317.691*X3+1635.079*X4+1324.686*X5−1467.609.

The discriminant equation for the cultured population is as follows:

F2=3428.068*X1−596.227*X2+2251.891*X3+1661.023*X4+1373.527*X5−1462.008.

In a specific embodiment, the step of determining the category of the golden pompano sample to be identified within Trachinotus anak based on the calculation result of the discriminant function equation includes the following operations.

Values of F1 and F2 for the golden pompano sample to be identified are calculated, and the category of the golden pompano sample to be identified within Trachinotus anak is assigned as a category of a corresponding discriminant equation associated with a relatively higher value between the values of F1 and F2.

In the specific embodiment, the above discriminant function equation is provided. A significance test is performed on the discriminant function equation, and a result shows P<0.01, indicating that the discriminant function equation reaches a significant level and can effectively distinguish between the wild population and the cultured population.

Incorporating all specific embodiments of the disclosure, an embodiment 2 of the disclosure includes the following step (1) through step (9).

In step (1), a golden pompano sample to be identified is collected.

In step (2), preliminary morphological identification, morphometric measurement, DR imaging, and DNA extraction are performed on the golden pompano sample to be identified. A body weight of the golden pompano sample to be identified is measured with an electronic balance. A photograph is taken of a left lateral side of the golden pompano sample to be identified by using a camera. The morphometric measurement is then performed by using a MSHOT™ image analysis system to obtain the following morphometric data: a fork length FL, a distance Sn-D1O from a snout tip to a first-dorsal-fin origin, a distance Sn-D2O from the snout tip to a second-dorsal-fin origin, a distance Sn-P2O from the snout tip to a pelvic-fin origin, a distance Sn-A2O from the snout tip to an anal-fin origin, a distance D1O-P2O from the first-dorsal-fin origin to the pelvic-fin origin, a distance D1O-A2O from the first-dorsal-fin origin to the anal-fin origin, a distance D2O-P2O from the second-dorsal-fin origin to the pelvic-fin origin, a distance D2O-A2O from the second-dorsal-fin origin to the anal-fin origin, a midbody depth MBD, a half body depth at a pectoral-fin origin P1-D2I, a second-dorsal-fin length HD2L, an anal-fin length HAL, a pectoral-fin length PIL, a pelvic-fin length P2L, a second-dorsal-fin base length LDB, an anal-fin base length LAB, a head length HL, a post-orbital head length PoHL, an eye diameter ED, a snout length SnL, an upper-jaw length UjL, and a maxilla depth MaD.

The above MSHOT™ image analysis system is a powerful software application for two-dimensional (2D) image processing and analysis, featuring comprehensive measurement capabilities.

A method for extracting DNA from the golden pompano sample to be identified is not particularly limited. Any conventional animal genomic DNA extraction method standard in the related art may be used. Specifically, a commercial extraction kit is used.

In step (3), the morphometric data (e.g., a ratio of the second-dorsal-fin length to the fork length) and a DR imaging result obtained in step (2) are consulted with descriptions in a dichotomous key, thereby determining from a morphological perspective whether the golden pompano sample to be identified is Trachinotus anak.

The DR imaging result is primarily examined for: shapes of first interneural supraoccipital bones; whether increased bone density is observed in preorbital and nasal bones; and whether ventral ribs are enlarged and exhibit increased bone density. The content of the dichotomous key is as follows.
1(2) Body oval; body silvery-white with multiple oval dark spots on sides; dorsal and anal fins white with black tips; caudal fin black with a white margin only . . . Trachinotus ovatus (eastern Atlantic)
2(1) Body rhomboidal; body golden-yellow with a grayish-yellow dorsal area, or body silvery-white with a grayish-green dorsal area; no spots on sides; dorsal and anal fins yellow without black tips; caudal fin yellow.
3(4) First interneural bone oval bead-shaped; anterior rays of second dorsal fin distinctly elongated, 1.5 to 2.0 times the head length, or 35% to 60% of the fork length for individuals of 100 millimeters (mm) to 400 mm . . . Trachinotus blochii (Indo-Pacific)
4(3) First interneural bone inverted L-shaped; anterior rays of both second dorsal and anal fins relatively short; the second-dorsal-fin length 24% to 30% of the fork length.
5(6) Ventral ribs not enlarged; no increase in bone density observed in preorbital and nasal bones; supraoccipital bone widened, appearing as an elongated elliptical cylinder in individuals with the fork length exceeding 200 mm, more pronounced in individuals with the fork length exceeding 300 mm . . . Trachinotus mookalee (Indo-Pacific)
6(5) Supraoccipital bone thin and blade-shaped; second or enlarged third ventral ribs enlarged; increased bone density observed in preorbital and nasal bones in individuals each with the fork length exceeding 300 mm . . . Trachinotus anak (Western Pacific)

In step (4), the DNA obtained in the step (2) is used as a DNA template, and then PCR amplification is performed with primers to obtain an amplification product. The amplification product is purified and sequenced to obtain a sequencing result, and the sequencing result is aligned.

The DNA is located in an COI region of mitochondrial DNA (mtDNA).

The primers for amplifying the DNA include a FishF1 primer and a FishR1 primer. The nucleotide sequence of the FishF1 primer is: 5′-TCAACCAACCACAAAGACATTGGCAC-3′. The nucleotide sequence of the FishR1 primer is: 3′-TAGACTTCTGGGTGGCCAAACAATCA-5′.

A system for the PCR amplification has a total volume of 25 μL and includes the following components: 2 μL of the DNA template, 12.5 μL of Taq PCR Mix, 6.5 μL of ddH₂O, 2 μL of MgCl₂, 1 μL of the FishF1 primer, and 1 μL of the FishR1 primer.

Procedures for the PCR amplification include: pre-denaturation at 95 °C for 3 minutes; 35 cycles of denaturation at 92 °C for 45 seconds, annealing at 50 °C for 45 seconds, and extension at 72 °C for 1 minute; and finally extension at 72 °C for 10 minutes, then hold at 4 °C.

Specifically, upon completion of the PCR amplification, purification and sequencing of the amplification product are performed. Methods for the purification and sequencing are not particularly limited and are specifically outsourced to a commercial biological sequencing company.

In step (5), a sequence of the amplification product is aligned with COI sequences downloaded from an NCBI database to construct a phylogenetic tree. When the sequence of the amplification product clusters into a clade with a COI sequence corresponding to Trachinotus anak in the phylogenetic tree, it is identified by molecular marker identification that the golden pompano sample to be identified is Trachinotus anak.

The COI sequences used for constructing the phylogenetic tree are as shown in SEQ ID NO: 1 to SEQ ID NO: 49. Specifically, SEQ ID NO: 1 to SEQ ID NO: 4 are the COI sequences from Trachinotus anak, SEQ ID NO: 5 to SEQ ID NO: 47 are the COI sequences from the genus Trachinotus, and SEQ ID NO: 48 and SEQ ID NO: 49 are the outgroup sequences required for constructing the phylogenetic.

In step (6), when results of both the step (3) and the step (5) indicate that the golden pompano sample to be identified is Trachinotus anak, the golden pompano sample to be identified is identified as Trachinotus anak.

In step (7), standardization is performed on the morphometric data obtained in the step (2) to obtain standardized morphometric data. Specifically, each of the post-orbital head length PoHL, the eye diameter ED, the snout length SnL, the upper-jaw length UjL, and the maxilla depth MaD is divided by the head length HL, and each of remaining morphometric data of the morphometric data is divided by the fork length FL.

In step (8), a statistical model is constructed from the standardized morphometric data obtained in the step (7) by using a Wilk's lambda method, and an F-distribution value is used as a criterion. A variable is introduced into the statistical model when a F-value of the variable is greater than 3.84, and a variable is removed when a F-value of the variable is less than 2.71. Through stepwise variable introduction, 22 standardized morphometric data are gradually introduced over 5 operational steps. Then, variables with small contribution rates to the statistical model are gradually eliminated according to their contribution rates. Ultimately, five variables are selected: the distance from the snout tip to the pelvic-fin origin divided by the fork length, denoted as X1; the half body depth at the pectoral-fin origin divided by the fork length, denoted as X2; the anal-fin base length divided by the fork length, denoted as X3; the post-orbital head length divided by the head length, denoted as X4; and the eye diameter divided by the head length, denoted as X5. Then, a collinearity analysis is performed on these five variables, and a result indicates that the collinearity among the five variables is very low and there is no collinearity issue. In addition, a discriminant function equation is constructed as follows, which includes a discriminant equation for the wild population and a discriminant equation for the cultured population.

Specifically, the discriminant equation for the wild population is constructed as follows:

F1=3504.230*X1−650.078*X2+2317.691*X3+1635.079*X4+1324.686*X5−1467.609.

The discriminant equation for the cultured population is constructed as follows:

F2=3428.068*X1−596.227*X2+2251.891*X3+1661.023*X4+1373.527*X5−1462.008.

Then, a significance test is performed on the discriminant function equation, and a result shows P<0.01, indicating that the discriminant function equation reaches a significant level and can effectively distinguish between the wild population and the cultured population.

In step (9), the morphometric data of the golden pompano sample to be identified is substituted into the discriminant equations, and a category of a corresponding discriminant function yielding a greater function value is determined as a category of the golden pompano sample to be identified within Trachinotus anak.

Specifically, when the disclosure is applied, the disclosure includes the following step 1 through step 6.

In step 1, a total of 108 golden pompano samples are collected, including 58 specimens from cultured population and 50 specimens from natural population. A body weight of each golden pompano sample is measured with an electronic balance. A photograph is taken of a left lateral side of each golden pompano sample by using a camera. An appropriate amount of tissue of each golden pompano sample is collected and preserved at −20 °C. Morphometric measurement is performed on each golden pompano sample by using a MSHOT™ image analysis system to obtain the following morphometric data: a fork length FL, a distance Sn-D1O from a snout tip to a first-dorsal-fin origin, a distance Sn-D2O from the snout tip to a second-dorsal-fin origin, a distance Sn-P2O from the snout tip to a pelvic-fin origin, a distance Sn-A2O from the snout tip to an anal-fin origin, a distance D1O-P2O from the first-dorsal-fin origin to the pelvic-fin origin, a distance D1O-A2O from the first-dorsal-fin origin to the anal-fin origin, a distance D2O-P2O from the second-dorsal-fin origin to the pelvic-fin origin, a distance D2O-A2O from the second-dorsal-fin origin to the anal-fin origin, a midbody depth MBD, a half body depth at a pectoral-fin origin P1-D2I, a second-dorsal-fin length HD2L, an anal-fin length HAL, a pectoral-fin length PIL, a pelvic-fin length P2L, a second-dorsal-fin base length LDB, an anal-fin base length LAB, a head length HL, a post-orbital head length PoHL, an eye diameter ED, a snout length SnL, an upper-jaw length UjL, and a maxilla depth MaD.

In step 2, the obtained morphometric data of some golden pompano samples show: the second-dorsal-fin divided by the fork length is less than 30%. The DR imaging results show: the first interneural bone is inverted L-shaped; the supraoccipital bone is blade-shaped; the second and third ventral ribs are enlarged; and increased bone density is observed in the preorbital and nasal bones of individuals (also referred to as specimens) with a fork length exceeding 300 mm.

By consulting descriptions in a dichotomous key, the samples to be identified in the step 2 are determined to be Trachinotus anak.

In step 3, DNA is extracted from each sample to be identified. Using the extracted DNA as a DNA template, PCR amplification is performed with primers to obtain an amplification product. The amplification product is purified and sequenced to obtain a sequencing result, and the sequencing result is aligned. The primers include a FishF1 primer and a FishR1 primer.

The sequence of the FishF1 primer is as shown in SEQ ID NO: 50: 5′-TCAACCAACCACAAAGACATTGGCAC-3′.

The sequence of the FishR1 primer is as shown in SEQ ID NO: 51: 3′-TAGACTTCTGGGTGGCCAAACAATCA-5′.

Into a 0.2 milliliters (mL) centrifuge tube, the following are added in sequence: 12.5 μL of Taq PCR Mix, 6.5 μL of ddH₂O, 2 μL of MgCl₂, 2 μL of the DNA template, and 1 μL each of the FishF1 primer and the FishR1 primer. Procedures for the PCR amplification include: pre-denaturation at 95 °C for 3 minutes; 35 cycles of denaturation at 92 °C for 45 seconds, annealing at 50 °C for 45 seconds, and extension at 72 °C for 1 minute; and finally extension at 72 °C for 10 minutes. The amplification product is sent to Sangon Biotech (Shanghai) Co., Ltd. for sequencing.

In step 4, after sequencing, the sequencing result is aligned, and a phylogenetic tree is constructed based on the sequencing result.

A result of the phylogenetic tree (referring to FIG. 4) shows that the sample to be identified clusters into a clade with Trachinotus anak.

In step 5, a combined result of the morphological analysis (step 2) and the sequencing result (step 4) indicates that the sample to be identified is Trachinotus anak.

In step 6, five morphometric data of each Trachinotus anak specimen to be identified are substituted into the discriminant equations, where an equation yielding a greater calculated value determines its belonging category. The discriminant results (as shown in Table 1 below) show that an identification accuracy rate for the wild population of Trachinotus anak is 80.0%, an identification accuracy rate for the cultured population is 86.2%, and an overall identification accuracy rate is 83.3%.

TABLE 1
Identification Accuracy Rate for the Wild and
Cultured Populations of Trachinotus anak

| | | | | | Overall |
|---|---|---|---|---|---|
| | | | | Identifi- | identifi- |
| | | Identification results | | cation | cation |
| | Number of | Wild | Cultured | accuracy | accuracy |
| Category | specimens | population | population | rate (%) | rate (%) |
| Wild | 50 | 40 | 10 | 80.0 | 83.3. |
| population | | | | | |
| Cultured | 58 | 8 | 50 | 86.2 | |
| population | | | | | |

The disclosure further provides a device for identifying Trachinotus anak and distinguishing between wild population and cultured population thereof, which includes a sample acquisition module, a data acquisition module, a first determination module, a DNA extraction module, a PCR amplification module, a second determination module, a species determination module, and a category determination module.

The sample acquisition module is configured to obtain a golden pompano sample to be identified.

The data acquisition module is configured to perform morphometric measurement on the golden pompano sample to be identified to obtain morphometric data of the golden pompano sample to be identified, and perform DR imaging on the golden pompano sample to be identified to obtain a DR imaging result of the golden pompano sample to be identified.

The first determination module is configured to determine, based on the morphometric data of the golden pompano sample to be identified, the DR imaging result of the golden pompano sample to be identified, and a pre-established dichotomous key, whether the golden pompano sample to be identified is Trachinotus anak, thereby to obtain a first determination result.

The DNA extraction module is configured to perform DNA extraction on the golden pompano sample to be identified to obtain DNA of the golden pompano sample to be identified.

The PCR amplification module is configured to perform, using the DNA of the golden pompano sample to be identified as a DNA template, PCR amplification with primers to obtain an amplification product of the golden pompano sample to be identified, and purify and sequence the amplification product to obtain a sequencing result of the golden pompano sample to be identified.

The second determination module is configured to align the sequencing result of the golden pompano sample to be identified with COI sequences downloaded from an NCBI database to construct a phylogenetic tree, and determine, based on the phylogenetic tree, whether the golden pompano sample to be identified is Trachinotus anak, thereby to obtain a second determination result.

The species determination module is configured to identify the golden pompano sample to be identified as Trachinotus anak in response to both the first determination result and the second determination result indicating that the golden pompano sample to be identified is Trachinotus anak.

The category determination module is configured to standardize the morphometric data to obtain standardized morphometric data of the golden pompano sample to be identified, substitute the standardized morphometric data into a discriminant function equation to obtain a calculation result, and determine, based on the calculation result of the discriminant function equation, a category of the golden pompano sample to be identified within Trachinotus anak, where the category includes the cultured population and the wild population.

In an embodiment, each of the sample acquisition module, the data acquisition module, the first determination module, the DNA extraction module, the PCR amplification module, the second determination module, the species determination module, and the category determination module is embodied by at least one processor and at least one memory coupled to the at least one processor, and the at least one memory stores computer programs executable by the at least one processor.

Although the specification of the disclosure has been sufficiently detailed and specifically described with respect to several of the described embodiments, it is not intended to be limited to any of these details or embodiments or any particular embodiments, but rather should be considered as providing a broad possible interpretation of the appended claims by reference to the appended claims and taking into account the related art, effectively covering the intended scope of the disclosure. In addition, the above description of the disclosure is based on embodiments foreseeable by the applicant, with the aim of providing a useful description. Non substantial modifications to the disclosure that have not yet been foreseen can still represent equivalent modifications of the disclosure.

The above descriptions are merely specific embodiments of the disclosure, and the disclosure is not limited to the above embodiments. As long as the same means is used to achieve the technical effects of the disclosure, it should be within the scope of protection of the disclosure. Within the scope of protection of the disclosure, the technical solution and/or implementation may have various modifications and variations.

What is claimed is:

1. A method for identifying Trachinotus anak and distinguishing between wild population and cultured population thereof, comprising:
obtaining a golden pompano sample to be identified;
performing morphometric measurement on the golden pompano sample to be identified to obtain morphometric data of the golden pompano sample to be identified, and performing digital radiography (DR) imaging on the golden pompano sample to be identified to obtain a DR imaging result of the golden pompano sample to be identified;
determining, based on the morphometric data of the golden pompano sample to be identified, the DR imaging result of the golden pompano sample to be identified, and a pre-established dichotomous key, whether the golden pompano sample to be identified is Trachinotus anak, thereby to obtain a first determination result;
performing deoxyribonucleic acid (DNA) extraction on the golden pompano sample to be identified to obtain DNA of the golden pompano sample to be identified;
performing, using the DNA of the golden pompano sample to be identified as a DNA template, polymerase chain reaction (PCR) amplification with primers to obtain an amplification product of the golden pompano sample to be identified, and purifying and sequencing the amplification product to obtain a sequencing result of the golden pompano sample to be identified;
aligning the sequencing result of the golden pompano sample to be identified with cytochrome c oxidase subunit I (COI) sequences downloaded from a national center for biotechnology information (NCBI) database to construct a phylogenetic tree, and determining, based on the phylogenetic tree, whether the golden pompano sample to be identified is Trachinotus anak, thereby to obtain a second determination result;
in response to both the first determination result and the second determination result indicating that the golden pompano sample to be identified is Trachinotus anak, identifying the golden pompano sample to be identified as Trachinotus anak; and
standardizing the morphometric data to obtain standardized morphometric data of the golden pompano sample to be identified, substituting the standardized morphometric data into a discriminant function equation to obtain a calculation result, and determining, based on the calculation result of the discriminant function equation, a category of the golden pompano sample to be identified within Trachinotus anak, wherein the category comprises the cultured population and the wild population.

2. The method for identifying Trachinotus anak and distinguishing between the wild population and the cultured population thereof as claimed in claim 1, wherein the morphometric data of the golden pompano sample to be identified comprises:
a fork length FL, a distance Sn-D1O from a snout tip to a first-dorsal-fin origin, a distance Sn-D2O from the snout tip to a second-dorsal-fin origin, a distance Sn-P2O from the snout tip to a pelvic-fin origin, a distance Sn-A2O from the snout tip to an anal-fin origin, a distance D1O-P2O from the first-dorsal-fin origin to the pelvic-fin origin, a distance D1O-A2O from the first-dorsal-fin origin to the anal-fin origin, a distance D2O-P2O from the second-dorsal-fin origin to the pelvic-fin origin, a distance D2O-A2O from the second-dorsal-fin origin to the anal-fin origin, a midbody depth MBD, a half body depth at a pectoral-fin origin P1-D2I, a second-dorsal-fin length HD2L, an anal-fin length HAL, a pectoral-fin length PIL, a pelvic-fin length P2L, a second-dorsal-fin base length LDB, an anal-fin base length LAB, a head length HL, a post-orbital head length PoHL, an eye diameter ED, a snout length SnL, an upper-jaw length UjL, and a maxilla depth MaD.

3. The method for identifying Trachinotus anak and distinguishing between the wild population and the cultured population thereof as claimed in claim 2, wherein the pre-established dichotomous key is specifically as follows:
1(2) body oval; body silvery-white with a plurality of oval dark spots on sides; dorsal and anal fins white with black tips; caudal fin black with a white margin only wherein the pre-established dichotomous key is in a continuous format, a number at a beginning of a line indicates an item number identifying morphological characters of a current item, and a number in parentheses indicates a target item to jump to when the morphological characters of the current item are not satisfied; and
wherein “1” is an item number, when morphological characters of the item 1 are met, a species of the golden pompano sample to be identified is determined as Trachinotus ovatus, when the morphological characters of the item 1 are not met, a process proceeds to item 2;
2(1) body rhomboidal; body golden-yellow with a grayish-yellow dorsal area, or body silvery-white with a grayish-green dorsal area; no spots on sides; dorsal and anal fins yellow without black tips; caudal fin yellow;
wherein “2” is an item number, when morphological characters of the item 2 are met, the process proceeds to item 3 to check remaining morphological characters, when the morphological characters of the item 2 are not met, the process proceeds to the item 1;
3(4) first interneural bone oval bead-shaped; anterior rays of second dorsal fin distinctly elongated, 1.5 to 2.0 times the head length, or 35% to 60% of the fork length for individuals of 100 millimeters (mm) to 400 mm wherein “3” is an item number, when morphological characters of the item 3 are met, the species of the golden pompano sample to be identified is determined as Trachinotus blochii, when the morphological characters of the item 3 are not met, the process proceeds to item 4;
4(3) first interneural bone inverted L-shaped; anterior rays of both second dorsal and anal fins relatively short; the second-dorsal-fin length 24% to 30% of the fork length;
wherein “4” is an item number, when morphological characters of the item 4 are met, the process proceeds to item 5 to check remaining morphological characters, when the morphological characters of item 4 are not met, the process proceeds to the item 3;
5(6) ventral ribs not enlarged; no increase in bone density observed in preorbital and nasal bones; supraoccipital bone widened, appearing as an elongated elliptical cylinder in individuals with the fork length exceeding 200 mm, more pronounced in individuals with the fork length exceeding 300 mm
wherein “5” is an item number, when morphological characters of the item 5 are met, the species of the golden pompano sample to be identified is determined as Trachinotus mookalee, when the morphological characters of the item 5 are not met, the process proceeds to item 6; and
6(5) supraoccipital bone thin and blade-shaped; second or enlarged third ventral ribs enlarged; increased bone density observed in preorbital and nasal bones in individuals with the fork length exceeding 300 mm
wherein “6” is an item number, when morphological characters of the item 6 are met, the species of the golden pompano sample to be identified is determined as Trachinotus anak, when the morphological characters of the item 6 are not met, the process proceeds to item 5.

4. The method for identifying Trachinotus anak and distinguishing between the wild population and the cultured population thereof as claimed in claim 1, wherein the primers used for the PCR amplification comprise:
a FishF1 primer, wherein the sequence of the FishF1 primer is as shown in SEQ ID NO: 50: 5′-TCAACCAACCACAAAGACATTGGCAC-3′; and
a FishR1 primer, wherein the sequence of the FishR1 primer is as shown in SEQ ID NO: 51: 3′-TAGACTTCTGGGTGGCCAAACAATCA-5′;
wherein a system for the PCR amplification comprises: 2 microliters (μL) of the DNA template, 12.5 μL of TaqPCR Mix, 6.5 μL of double-distilled water (ddH₂O), 2 μL of magnesium chloride (MgCl₂), 1 μL of the FishF1 primer, and 1 μL of the FishR1 primer; and
wherein procedures for the PCR amplification comprise: pre-denaturation at 95 °C for 3 minutes; 35 cycles of denaturation at 92 °C for 45 seconds, annealing at 50 °C for 45 seconds, and extension at 72 °C for 1 minute; and finally extension at 72 °C for 10 minutes.

5. The method for identifying Trachinotus anak and distinguishing between the wild population and the cultured population thereof as claimed in claim 1, wherein the aligning the sequencing result of the golden pompano sample to be identified with COI sequences downloaded from an NCBI database to construct a phylogenetic tree, and determining, based on the phylogenetic tree, whether the golden pompano sample to be identified is Trachinotus anak, thereby to obtain a second determination result comprises:
aligning a sequence of the amplification product in the sequencing result of the golden pompano sample to be identified with the COI sequences downloaded from the NCBI database to construct the phylogenetic tree, and in response to the sequence of the amplification product clustering into a clade with a COI sequence corresponding to Trachinotus anak in the phylogenetic tree, identifying, by molecular marker identification, the golden pompano sample to be identified as Trachinotus anak.

6. The method for identifying Trachinotus anak and distinguishing between the wild population and the cultured population thereof as claimed in claim 2, wherein the standardizing the morphometric data of the golden pompano sample to be identified to obtain standardized morphometric data comprises:
dividing each of the post-orbital head length PoHL, the eye diameter ED, the snout length SnL, the upper-jaw length UjL, and the maxilla depth MaD by the head length HL, and dividing each of remaining morphometric data of the morphometric data by the fork length FL.

7. The method for identifying Trachinotus anak and distinguishing between the wild population and the cultured population thereof as claimed in claim 6, wherein the discriminant function equation is constructed based on a stepwise discriminant method, specifically comprising:
constructing a statistical model from the standardized morphometric data by using a Wilks' lambda method, using an F-distribution value as a criterion, introducing a variable into the statistical model when a F-value of the variable is greater than 3.84, and removing a variable when a F-value of the variable is less than 2.71, thereby ultimately screening out the following five standardized morphometric data from the standardized morphometric data: the distance from the snout tip to the pelvic-fin origin divided by the fork length, denoted as X1; the half body depth at the pectoral-fin origin divided by the fork length, denoted as X2; the anal-fin base length divided by the fork length, denoted as X3; the post-orbital head length divided by the head length, denoted as X4; and the eye diameter divided by the head length, denoted as X5; and
wherein the discriminant function equation comprises a discriminant equation for the wild population and a discriminant equation for the cultured population;
wherein the discriminant equation for the wild population is as follows:
F1=3504.230*X1−650.078*X2+2317.691*X3+1635.079*X4+1324.686*X5−1467.609;
wherein the discriminant equation for the cultured population is as follows:
F2=3428.068*X1−596.227*X2+2251.891*X3+1661.023*X4+1373.527*X5−1462.008.

8. The method for identifying Trachinotus anak and distinguishing between the wild population and the cultured population thereof as claimed in claim 7, wherein the determining, based on the calculation result of the discriminant function equation, a category of the golden pompano sample to be identified within Trachinotus anak comprises:
calculating values of F1 and F2 for the golden pompano sample to be identified, and assigning the category of the golden pompano sample to be identified within Trachinotus anak as a category of a corresponding discriminant equation associated with a higher value between the values of F1 and F2.

9. A device for identifying Trachinotus anak and distinguishing between wild population and cultured population thereof, comprising:
a sample acquisition module, configured to obtain a golden pompano sample to be identified;
a data acquisition module, configured to perform morphometric measurement on the golden pompano sample to be identified to obtain morphometric data of the golden pompano sample to be identified, and perform DR imaging on the golden pompano sample to be identified to obtain a DR imaging result of the golden pompano sample to be identified;
a first determination module, configured to determine, based on the morphometric data of the golden pompano sample to be identified, the DR imaging result of the golden pompano sample to be identified, and a pre-established dichotomous key, whether the golden pompano sample to be identified is Trachinotus anak, thereby to obtain a first determination result;
a DNA extraction module, configured to perform DNA extraction on the golden pompano sample to be identified to obtain DNA of the golden pompano sample to be identified;
a PCR amplification module, configured to perform, using the DNA of the golden pompano sample to be identified as a DNA template, PCR amplification with primers to obtain an amplification product of the golden pompano sample to be identified, and purify and sequence the amplification product to obtain a sequencing result of the golden pompano sample to be identified;
a second determination module, configured to align the sequencing result of the golden pompano sample to be identified with COI sequences downloaded from an NCBI database to construct a phylogenetic tree, and determine, based on the phylogenetic tree, whether the golden pompano sample to be identified is Trachinotus anak, thereby to obtain a second determination result;
a species determination module, configured to identify the golden pompano sample to be identified as Trachinotus anak in response to both the first determination result and the second determination result indicating that the golden pompano sample to be identified is Trachinotus anak; and
a category determination module, configured to standardize the morphometric data to obtain standardized morphometric data of the golden pompano sample to be identified, substitute the standardized morphometric data into a discriminant function equation to obtain a calculation result, and determine, based on the calculation result of the discriminant function equation, a category of the golden pompano sample to be identified within Trachinotus anak, where the category comprises the cultured population and the wild population.


SEQUENCE LISTING (51 sequences)

SEQ ID NO: 1 - DNA, 512 bp, Trachinotus anak
  source 1..512: mol_type = genomic DNA
agtcaacccggcgccctcctaggggatgaccaaatttacaatgtaatcgttacagcccacgccttcgtaatgattttctttatagtaatgccaattatgattggaggctttggaaactgacttatcccactaatgatcggagccccggatatggcattccctcgaataaataacatgagtttttgactcctacccccctctttccttctccttcttgcctcctcaggggtagaagcgggggccggaacaggttgaacagtttatccccccttagctggtaatcttgcccatgcaggagcatctgttgatttaaccattttctcgcttcatctagctggaatctcatcaattctaggagctattaacttcatcacaacagtaattaacataaaacctcatgctgtatctatatatcaaattccactatttgtctgagccgttctaattacagccgtccttctgctcctctcacttcctgttttagccgccggcattacaatacttctaaccga

SEQ ID NO: 2 - DNA, 512 bp, Trachinotus anak
  source 1..512: mol_type = genomic DNA
agtcaacccggcgccctcctaggggatgaccaaatttacaatgtaatcgttacagcccacgccttcgtaatgattttctttatagtaatgccaattatgattggaggctttggaaactgacttatcccactaatgatcggagccccggatatggcattccctcgaataaataacatgagtttttgactcctacccccctctttccttctccttcttgcctcctcaggggtagaagcgggggccggaacaggttgaacagtttatccccccttagctggtaatcttgcccatgcaggagcatctgttgatttaaccattttctcgcttcatctagctggaatctcatcaattctaggagctattaacttcatcacaacagtaattaacataaaacctcatgctgtatctatatatcagattccactatttgtctgagccgttctaattacagccgtccttctgctcctctcacttcctgttttagccgccggcattacaatacttctaaccga

SEQ ID NO: 3 - DNA, 512 bp, Trachinotus anak
  source 1..512: mol_type = genomic DNA
agtcaacccggcgccctcctaggggatgaccaaatttacaatgtaatcgttacagcccacgccttcgtaatgattttctttatagtaatgccaattatgattggaggctttggaaactgacttattccactaatgatcggagccccggatatggcattccctcgaataaataacatgagtttttgactcctacccccctctttccttctccttcttgcctcctcaggggtagaagcgggggccggaacaggttgaacagtttatccccccttagctggtaatcttgcccatgcaggagcatctgttgatttaaccattttctcgcttcatctagctggaatctcatcaattctaggagctattaacttcatcacaacagtaattaacataaaacctcatgctgtatctatatatcaaattccactatttgtctgagccgttctaattacagccgtccttctgctcctctcacttcctgttttagccgccggcattacaatacttctaaccga

SEQ ID NO: 4 - DNA, 512 bp, Trachinotus anak
  source 1..512: mol_type = genomic DNA
agtcaacccggcgccctcctaggggatgaccaaatttacaatgtaatcgttacagcccacgccttcgtaatgattttctttatagtaatgccaattatgattggaggctttggaaactgacttatcccactaatgatcggagccccggatatggcattccctcgaataaataacatgagtttttgactcctgcccccctctttccttctccttcttgcctcctcaggggtagaagcgggggccggaacaggttgaacagtttatccccccttagctggtaatcttgcccatgcaggagcatctgttgatttaaccattttctcgcttcatctagctggaatctcatcaattctaggagctattaacttcatcacaacagtaattaacataaaacctcatgctgtatctatatatcaaattccactatttgtctgagccgttctaattacagccgtccttctgctcctctcacttcctgttttagccgccggcattacaatacttctaaccga

SEQ ID NO: 5 - DNA, 512 bp, Trachinotus ovatus; identical to SEQ ID NO: 1
  source 1..512: mol_type = genomic DNA

SEQ ID NO: 6 - DNA, 512 bp, Trachinotus ovatus; identical to SEQ ID NO: 1
  source 1..512: mol_type = genomic DNA

SEQ ID NO: 7 - DNA, 512 bp, Trachinotus anak; identical to SEQ ID NO: 3
  source 1..512: mol_type = genomic DNA

SEQ ID NO: 8 - DNA, 512 bp, Trachinotus anak
  source 1..512: mol_type = genomic DNA
agtcaacccggcgccctcctaggagatgaccaaatttacaatgtaatcgttacagcccacgccttcgtaatgattttctttatagtaatgccaattatgattggaggctttggaaactgacttattccactaatgatcggagccccggatatggcattccctcgaataaataacatgagtttttgactcctacctccctctttccttctccttcttgcctcctcaggggtagaagcgggggccggaacaggttgaacagtttatccccccttagctggtaatcttgcccatgcaggagcatctgttgatttaaccattttctcgcttcatctagctggaatctcatcaattctaggagctattaacttcatcacaacagtaattaacataaaacctcatgctgtatctatatatcaaattccactatttgtctgagccgttctaattacagccgtccttctgctcctctcacttcctgttttagccgccggcattacaatacttctaaccga

SEQ ID NO: 9 - DNA, 512 bp, Trachinotus ovatus
  source 1..512: mol_type = genomic DNA
agccaacccggcgccctcctaggagatgaccaaatttataatgtaatcgttacggcccatgctttcgtaatgattttctttatagtaataccaattatgattggaggctttggaaactgacttattcctctaatgatcggagcccctgatatagcattccctcgaatgaacaatatgagcttctgacttctacccccttctttcctacttcttctcgcttcctcaggagtagaagcaggtgccggaaccggatgaacagtctaccctcccctagctggaaaccttgctcatgcgggagcatctgttgatttaaccatcttctctctccacttagctggaatttcctcaattcttggggcaattaactttattacaacagtaattaacatgaaacctcatgccgtttccatgtaccaaattccactattcgtttgagctgtactaattacagctgttctccttcttctctcacttcccgtcctagctgcaggcattacaatgctccttacgga

SEQ ID NO: 10 - DNA, 512 bp, Trachinotus paitensis
  source 1..512: mol_type = genomic DNA
agtcaacccggcgccctcctaggagatgaccaaatttacaatgtaatcgttacagcccacgccttcgtaatgattttctttatagtaatgccaattatgattggaggcttcggaaactgacttattccactgatgatcggagcccctgatatagcattccctcgaataaataacatgagcttctgacttctccctccctctttccttcttcttctcgcctcctcaggggtagaggcaggtgccggaaccggttgaacagtttaccctcccttagctggtaaccttgcccatgcaggagcatctgtcgatttaaccattttctctcttcatttagctggtatttcatcaattctaggagctattaatttcatcacaacagtaatcaacatgaaacctcatgctgtctctatatatcaaatcccactatttgtctgagccgtcctaattacagctgtcctcctgctcctctcacttcccgttttagctgccggcattactatacttctaaccga

SEQ ID NO: 11 - DNA, 512 bp, Trachinotus goreensis
  source 1..512: mol_type = genomic DNA
agccaacctggcgccctcctaggagatgaccaaatttataatgtaatcgttactgcccatgccttcgtaatgatcttctttatagtaatgccaattatgattggaggctttgggaattgactcatcccactaatgatcggagcccctgacatagcattcccccgaatgaataacatgagcttctgacttctccctccctctttccttctccttcttgcctcctcaggagtagaagcaggtgccggaactggttgaacagtctaccctcctttagctggcaacctcgctcatgcaggagcatctgttgatttaaccatcttctctcttcacctggctgggatttcctcaattctaggggccattaatttcatcacaacagtaatcaacatgaagcctcacgcggtctcaatgtatcaaatcccattatttgtttgagccgtcctaattacagctgtacttctgctcctctcacttcccgtcctagctgccggcatcacaatgctcctaaccga

SEQ ID NO: 12 - DNA, 512 bp, Trachinotus goreensis; identical to SEQ ID NO: 11
  source 1..512: mol_type = genomic DNA

SEQ ID NO: 13 - DNA, 512 bp, Trachinotus carolinus
  source 1..512: mol_type = genomic DNA
agtcaacctggcgccctcctaggagatgaccaaatttacaatgtaatcgttacagctcatgccttcgtaatgattttctttatagtaatgccaattatgattgggggctttggaaactgacttatcccactaatgattggagcccctgatatagcattccctcgaataaataacatgagcttctgacttctccctccctctttccttcttcttctcgcctcctctggggtagaagcaggtgccggaaccggttgaacagtctaccctcccttagctggtaatcttgcccatgcaggagcatctgttgatttaaccattttctctcttcatttagctggtatttcatcaattctaggggctattaacttcatcacaacagtaattaacataaaacctcatgctgtctctatatatcaaatcccactatttgtctgagccgttctaatcacagctgtcctcctgctcctctcacttcccgttttagctgccggcattactatgcttctaaccga

SEQ ID NO: 14 - DNA, 512 bp, Trachinotus baillonii
  source 1..512: mol_type = genomic DNA
agccaacccggcgccctcttaggagatgaccaaatttacaacgtaatcgtcacggcccatgccttcgtgatgattttctttatagtaataccaattatgatcggaggctttggaaactgacttatccccttaatgatcggagcccctgatatagcatttcctcgaatgaacaatatgagcttctgacttctacccccttctttccttctcctcctcgcttcctcaggagtagaagcaggtgccgggactggttgaacagtctaccctcccctggctggaaacctcgctcatgcaggagcatctgttgacctaactattttctccctccacttagctggaatttcctcaattcttggagcaattaactttattacaacagtaattaacataaaacctcatgctgtttccatgtatcagattccactatttgtttgagctgtcctaattacagccgttcttctcctcctctcactccccgtcctagctgcaggcattacaatgcttcttacgga

SEQ ID NO: 15 - DNA, 512 bp, Trachinotus coppingeri
  source 1..512: mol_type = genomic DNA
agccaacccggcgccctcttaggagatgaccaaatttacaacgtaatcgttacggcccatgccttcgtaatgattttctttatagtaatgccaattatgatcggaggctttggaaactgacttatccccctaatgattggggcccctgatatagcatttcctcgaatgaacaatatgagcttctgacttctgcccccttctttccttcttcttctcgcttcctcaggagtagaagcaggtgctggaactggttgaacagtttaccctcccctggctggaaaccttgctcatgcgggagcatctgttgatctaactattttctcccttcacttagccggaatttcctcaattcttggagcaatcaactttattacaacagtaattaacataaaaccccatgctgtttccatgtaccaaattccactatttgtttgagctgttctaattacggctgtccttcttctcctctcactccccgttctagctgcaggcattacaatgcttcttacgga

SEQ ID NO: 16 - DNA, 512 bp, Trachinotus baillonii; identical to SEQ ID NO: 14
  source 1..512: mol_type = genomic DNA

SEQ ID NO: 17 - DNA, 512 bp, Trachinotus coppingeri
  source 1..512: mol_type = genomic DNA
agccaacccggcgccctcttaggagatgaccaaatttacaacgtaatcgttacggcccatgccttcgtaatgattttctttatagtaatgccaattatgatcggaggctttggaaactgacttatccccctaatgattggggcccctgatatagcatttcctcgaatgaacaatatgagcttctgacttctgcccccttctttccttcttcttctcgcttcctcaggagtagaagcaggtgctggaactggttgaacagtttaccctcccctggctggaaaccttgctcatgcgggagcatctgttgatctaactattttctcccttcacttagccggaatttcctcaattcttggagcaatcaactttattacaacagtaattaacataaaaccccatgctgtttccatgtaccaaattccactatttgtttgagctgttctaattacggccgtccttcttctcctctcactccccgttctagctgcaggcattacaatgcttcttacgga

SEQ ID NO: 18 - DNA, 512 bp, Trachinotus coppingeri; identical to SEQ ID NO: 15
  source 1..512: mol_type = genomic DNA

SEQ ID NO: 19 - DNA, 512 bp, Trachinotus baillonii; identical to SEQ ID NO: 14
  source 1..512: mol_type = genomic DNA

SEQ ID NO: 20 - DNA, 512 bp, Trachinotus mookalee
  source 1..512: mol_type = genomic DNA
agtcaacccggcgccctcctaggagatgaccaaatttacaacgtaatcgttacagcccatgccttcgtaatgattttctttatagtaatgccaattatgattggaggctttggaaactgacttattccgctaatgattggggcccctgatatggcattccctcgaatgaataacatgagtttttgactcctccctccctcttttcttctccttcttgcctcctcaggggtagaagcgggtgccggaaccggttgaacagtttatccccctttagctggtaatcttgcccatgcaggagcatctgttgatttaaccattttctcgcttcacttagctggaatttcatcaattctaggagctattaacttcatcacaacagtaattaacataaaacctcatgctgtttctatatatcaaatcccactatttgtctgagccgttctaattacagccgtccttctgctcctctcgcttcctgttttagccgccggcattacaatactcctaaccga

SEQ ID NO: 21 - DNA, 512 bp, Trachinotus mookalee
  source 1..512: mol_type = genomic DNA
agtcaacccggcgccctcctaggagatgaccaaatttacaacgtaatcgttacagcccatgccttcgtaatgattttctttatagtaatgccaattatgattggaggctttggaaactgacttattccgctaatgattggggcccctgatatggcattcccccgaatgaataacatgagtttttgactcctccctccctcttttcttctccttcttgcctcctcaggggtagaagcgggtgccggaaccggttgaacggtttatccccctttagctggtaatcttgcccatgcaggagcatctgttgatttaaccattttctcgcttcacttagctggaatttcatcaattttaggagctattaacttcatcacaacagtaattaacataaaacctcatgctgtttctatatatcaaattccactatttgtctgagccgttctaattacagccgtccttctgctcctctcacttcctgttttagccgccggcattacaatactcctaaccga

SEQ ID NO: 22 - DNA, 512 bp, Trachinotus mookalee
  source 1..512: mol_type = genomic DNA
agtcaacccggcgcccttctaggagatgaccaaatttacaacgtaatcgttacagcccatgccttcgtaatgattttctttatagtaatgccaattatgattggaggctttggaaactgacttattccgctaatgattggagcccctgatatggcattccctcgaatgaataacatgagtttttgactcctccctccctcttttcttctccttcttgcctcctcaggggtagaagcgggtgccggaaccggttgaacagtttatccccctttagctggtaatcttgcccatgcaggagcatctgttgatttaaccattttctcgcttcacttagctggaatttcatcaattctaggagctattaacttcatcacaacagtaattaacataaaacctcatgctgtttctatatatcaaatcccactatttgtctgagccgttctaattacagccgtccttctgctcctctcgcttcctgttttagccgccggcattacaatactcctaaccga

SEQ ID NO: 23 - DNA, 512 bp, Trachinotus falcatus
  source 1..512: mol_type = genomic DNA
agtcaacccggcgcccttctaggggatgaccaaatttacaacgtaatcgttacagcccatgccttcgtaatgattttctttatagtaatgccaattatgatcggaggctttggaaactgactcattccactaatgatcggagcccccgatatggcattccctcggatgaacaacatgagcttttgacttcttcctccctcttttcttcttcttctcgcctcatcaggggtagaagcaggtgccggaaccggttgaacagtctatcctcccttagctggtaatcttgcccatgctggagcatctgttgatttaaccattttctcccttcacttagctggaatttcatcaatcctaggagctattaacttcattacaacagtaattaacataaaacctcatgctgtctctatatatcaaattccactatttgtctgagctgtcctaattacagccgtccttctgctcctctcactccctgttttagccgccggtattacaatacttttaaccga

SEQ ID NO: 24 - DNA, 512 bp, Trachinotus stilbe
  source 1..512: mol_type = genomic DNA
agtcaacccggcgccctcctaggggatgaccaaatttataatgtaatcgttacagctcatgccttcgtaatgattttctttatagtaataccaattatgattggaggctttggaaactgactcatcccactaatgattggggctcctgacatagccttccctcgaatgaacaatatgagcttctgactccttcccccctcttttcttctcctccttgcctcctcaggggtagaagcaggtgccggaaccggttggacagtctaccctccgctagctggtaatcttgcccacgcaggagcatcagttgatttaaccatcttctccttacacttagctggaatttcatcaattctaggggccattaacttcattactacagtaatcaacataaaaccccacgctgtttccatatatcaaattccactattcgtgtgagctgtcctaattacagctgtccttctgctcctctcactccccgtattagccgccggcattacaatacttcttaccga

SEQ ID NO: 25 - DNA, 512 bp, Trachinotus baillonii; identical to SEQ ID NO: 14
  source 1..512: mol_type = genomic DNA

SEQ ID NO: 26 - DNA, 512 bp, Trachinotus macrospilus
  source 1..512: mol_type = genomic DNA
agccaacccggcgccctcttaggagatgaccaaatttataacgtaatcgttacggcccatgccttcgtaatgattttctttatagtaataccaattatgatcggaggctttggaaactgacttatccccttaatgatcggagcccctgatatagcatttcctcgaatgaacaatatgagcttctgacttctacccccttctttcctcctcctcctcgcttcctcaggagtagaagcaggtgccgggactggttgaacagtctaccctcccctggctggaaaccttgctcatgcaggagcatccgttgacctaactattttctcccttcacttagctggaatttcctcaattcttggagcaattaactttattacaacagtaattaacataaaaccccatgctgtttccatgtatcaaattccgctatttgtttgagctgtcctaattacagccgttctcctcctcctctcactccccgtcctagctgcaggcattacaatgcttcttacgga

SEQ ID NO: 27 - DNA, 512 bp, Trachinotus macrospilus
  source 1..512: mol_type = genomic DNA
agccaacccggcgccctcttaggagatgaccaaatttataacgtaatcgttacggcccatgccttcgtaatgattttctttatagtaataccaattatgatcggaggctttggaaactgacttatccccttaatgatcggagcccctgatatagcatttcctcgaatgaacaatatgagcttctgacttctacccccttctttcctcctcctcctcgcttcctcaggagtagaagcaggtgccgggactggttgaacagtctaccctcccctggctggaaaccttgctcatgcaggagcatccgttgacctaactattttctccctccacttagctggaatttcctcaattcttggagcaattaactttattacaacagtaattaacataaaaccccatgctgtttccatgtatcaaattccgctatttgtttgagctgtcctaattacagccgttctcctcctcctctcactccccgtcctagctgcaggcattacaatgcttcttacgga

SEQ ID NO: 28 - DNA, 512 bp, Trachinotus baillonii; identical to SEQ ID NO: 14
  source 1..512: mol_type = genomic DNA

SEQ ID NO: 29 - DNA, 512 bp, Trachinotus falcatus
  source 1..512: mol_type = genomic DNA
agtcaacccggcgcccttctaggggatgaccaaatttacaacgtaatcgttacagcccatgccttcgtaatgattttctttatagtaatgccaattatgatcggaggctttggaaactgactcattccactaatgatcggagcccccgatatggcattccctcggatgaacaacatgagcttttgacttcttcctccctcttttcttcttctcctcgcctcatcaggggtagaagcaggtgccggaaccggttgaacagtctatcctcccttagctggtaatcttgcccatgctggagcatctgttgatttaaccattttctcccttcacttagctggaatttcatcaatcctaggagctattaacttcattacaacagtaattaacataaaacctcatgctgtctctatatatcaaattccactatttgtctgagctgtcctaattacagccgtccttctgctcctctcactccctgttttagccgccggtattacaatacttttaaccga

SEQ ID NO: 30 - DNA, 512 bp, Trachinotus falcatus; identical to SEQ ID NO: 23
  source 1..512: mol_type = genomic DNA

SEQ ID NO: 31 - DNA, 512 bp, Trachinotus goodei
  source 1..512: mol_type = genomic DNA
agtcaacccggcgccctcctaggagatgaccaaatttacaatgtaatcgttacagcccatgccttcgtaatgattttctttatagtaataccaattatgattggaggctttggaaactgacttattccactaatgattggagctccagatatggcattccctcgaatgaataacatgagcttctgactcctccctccctcttttcttctccttcttgcctcctcaggagtagaagcaggtgccggaaccggttgaacagtctatccccccttagctggtaatcttgcccatgcaggagcatccgttgatttaaccattttctcccttcacctagctggaatttcatcaattctaggggctatcaacttcattacgacagtaatcaacataaaaccccatgctgtctctatataccagatcccactatttgtctgagccgttctaattacagccgtcctcctgcttctctcactacctgttttagccgctggcattacaatacttcttactga

SEQ ID NO: 32 - DNA, 512 bp, Trachinotus goodei; identical to SEQ ID NO: 31
  source 1..512: mol_type = genomic DNA

SEQ ID NO: 33 - DNA, 512 bp, Trachinotus rhodopus
  source 1..512: mol_type = genomic DNA
agtcaacccggcgccctcctaggagatgaccaaatttacaatgtaatcgttacagcccatgccttcgtaatgattttctttatagtaatgccaattatgattggaggctttggaaactgacttattccactaatgattggagctccagatatggcattccctcgaatgaataacatgagcttctgactcctccctccctcttttcttctccttcttgcctcctcaggagtagaagcaggtgccggaaccggttgaacagtctatccccccttagctggtaatcttgcccatgcaggagcatccgttgatttaaccattttctcccttcacttagctggaatttcatcaattctaggggctatcaacttcattacaacagtaatcaatatgaaaccccatgctgtctctatataccaaatcccactatttgtctgagccgttctaattacagccgtactcctgcttctctcgctacctgttttagccgctggcattacaatacttcttactga

SEQ ID NO: 34 - DNA, 512 bp, Trachinotus baillonii; identical to SEQ ID NO: 14
  source 1..512: mol_type = genomic DNA

SEQ ID NO: 35 - DNA, 512 bp, Trachinotus carolinus
  source 1..512: mol_type = genomic DNA
agtcaacctggcgccctcctaggagatgaccaaatttacaatgtaatcgttacagctcatgccttcgtaatgattttctttatagtaatgccaattatgattggaggctttggaaactgacttatcccactaatgattggagcccctgatatagcattccctcggataaataacatgagcttctgacttctccctccctctttccttcttcttctcgcctcctctggggtagaagcaggtgccggaaccggttgaacagtctaccctcccttagctggtaatcttgcccatgcaggagcatctgttgatttaaccattttctctcttcatttagctggtatttcatcaattctaggggctattaacttcatcacaacagtaattaacataaaacctcatgctgtctctatatatcaaatcccactatttgtctgagccgttctaatcacagctgtcctcctgctcctctcacttcccgttttagctgccggcattactatgcttctaaccga

SEQ ID NO: 36 - DNA, 512 bp, Trachinotus carolinus
  source 1..512: mol_type = genomic DNA
agtcaacctggcgccctcctaggagatgaccaaatttacaatgtaatcgttacagctcatgccttcgtaatgattttctttatagtaatgccaattatgattggaggctttggaaactgacttatcccactaatgattggagcccctgatatagcattccctcgaataaataacatgagcttctgacttctccctccctctttccttcttcttctcgcctcctctggggtagaagcaggtgccggaaccggttgaacagtctaccctcccttagctggtaatcttgcccatgcaggagcatctgttgatttaaccattttctctcttcatttagctggtatttcatcaattctaggggctattaacttcatcacaacagtaattaacataaaacctcatgctgtctctatatatcaaatcccactatttgtctgagccgttctaatcacagctgtcctcctgctcctctcacttcccgttttagctgccggcattactatgcttctaaccga

SEQ ID NO: 37 - DNA, 512 bp, Trachinotus botla
  source 1..512: mol_type = genomic DNA
agtcaacctggcgccctcctaggagatgaccaaatttataatgtaattgttacggcccatgccttcgtaatgattttctttatagtaatgccaattatgatcggaggctttggaaactgactcattccactaatgatcggagcccccgatatagccttcccccgtatgaacaacatgagcttctgacttctgcccccttctttccttcttcttctcgcttcctcaggagtagaagcaggtgccggaactggttgaacagtctaccctcccctggctggaaaccttgcccatgcaggagcatctgttgatttaaccattttctcccttcacttagctggaatttcctcaattcttggagcaattaactttattacaacagtaatcaatataaaaccccatgctgtttccatataccaaattccactatttgtttgagctgttctaattacagctgttcttcttcttctctctcttcctgtcctagctgcaggcattacaatgcttcttacgga

SEQ ID NO: 38 - DNA, 512 bp, Trachinotus botla
  source 1..512: mol_type = genomic DNA
agtcaacctggcgccctcctaggagatgaccaaatttataatgtaattgttacggcccatgccttcgtaatgattttctttatagtaatgccaattatgatcggaggctttggaaactgacttattccactaatgatcggagcccccgatatagccttcccccgtatgaacaacatgagcttctgacttctgcccccttctttccttcttcttctcgcttcctcaggagtagaagcaggtgccggaactggttgaacagtctaccctcccctggctggaaaccttgcccatgcaggagcatctgttgatttaaccattttctcccttcacttagctggaatttcctcaattcttggagcaattaactttattacaacagtaatcaatataaaaccccatgctgtttccatataccaaattccactatttgtttgagctgttctaattacagctgttcttcttcttctctctcttcctgtcctagctgcaggcattacaatgcttcttacgga

SEQ ID NO: 39 - DNA, 512 bp, Trachinotus botla; identical to SEQ ID NO: 38
  source 1..512: mol_type = genomic DNA

SEQ ID NO: 40 - DNA, 512 bp, Trachinotus botla; identical to SEQ ID NO: 38
  source 1..512: mol_type = genomic DNA

SEQ ID NO: 41 - DNA, 512 bp, Trachinotus baillonii
  source 1..512: mol_type = genomic DNA
agccaacccggcgccctcttaggagatgaccaaatttacaacgtaatcgtcacggcccatgccttcgtgatgattttctttatagtaataccaattatgatcggaggctttggaaactgacttatccccttaatgatcggagccccngatatagcatttcctcgaatgaacaatatgagcttctgacttctacccccttctttccttctcctcctcgcttcctcaggagtagaagcaggtgccgggactggttgaacagtctaccctcccctggctggaaacctcgctcatgcaggagcatctgttgacctaactattttctccctccacttagctggaatttcctcaattcttggagcaattaactttattacaacagtaattaacataaaacctcatgctgtttccatgtatcagattccactatttgtttgagctgtcctaattacagccgttcttctcctcctctcactccccgtcctagctgcaggcattacaatgcttcttacgga

SEQ ID NO: 42 - DNA, 512 bp, Trachinotus ovatus; identical to SEQ ID NO: 9
  source 1..512: mol_type = genomic DNA

SEQ ID NO: 43 - DNA, 512 bp, Trachinotus ovatus; identical to SEQ ID NO: 9
  source 1..512: mol_type = genomic DNA

SEQ ID NO: 44 - DNA, 512 bp, Trachinotus ovatus; identical to SEQ ID NO: 9
  source 1..512: mol_type = genomic DNA

SEQ ID NO: 45 - DNA, 512 bp, Trachinotus blochii
  source 1..512: mol_type = genomic DNA
agtcaacccggcgccctcctaggagatgaccaaatttataacgtaatcgttacagcccatgccttcgtaatgattttctttatagtaatgccaattatgattggaggctttggaaattgacttattccactaatgatcggagcccccgatatggcattccctcggatgaataacatgagtttttgactcctccctccctctttccttctcctactcgcctcctcaggagtagaagcaggtgctggaaccggttgaacggtgtacccacccttagctggtaatcttgcccatgcaggagcgtctgttgatttaactattttctctcttcatttagctggtatctcatcaattctaggggcaatcaacttcattacaacagtaatcaacatgaaaccccatgccgtctctatataccaaattccactatttgtctgagccgtcctaattacagctgtccttctgctcctctcacttcctgtcttagctgccggcattacaatacttttaaccga

SEQ ID NO: 46 - DNA, 512 bp, Trachinotus blochii
  source 1..512: mol_type = genomic DNA
agtcaacccggcgccctcctaggagatgaccaaatttataacgtaatcgttacagcccatgccttcgtgatgattttctttatagtaatgccaattatgattggaggctttggaaattgacttattccactaatgatcggagcccccgatatggcattccctcggatgaataacatgagtttttgactcctccctccctctttccttctcctactcgcctcctcaggagtagaagcaggtgctggaaccggttgaacggtgtacccacccttagctggtaatcttgcccatgcaggagcgtctgttgatttaactattttctctcttcatttagctggtatctcatcaattctaggggcaatcaacttcattacaacagtaatcaacatgaaaccccatgccgtctctatataccaaattccactatttgtctgagccgtcctaattacagctgtccttctgctcctctcacttcctgtcttagctgccggcattacaatacttttaaccga

SEQ ID NO: 47 - DNA, 512 bp, Trachinotus blochii
  source 1..512: mol_type = genomic DNA
agtcaacccggcgccctcctaggagatgaccaaatttataacgtaatcgttacagcccatgccttcgtgatgattttctttatagtaatgccaattatgattggaggctttggaaattgacttattccactaatgatcggagcccccgatatggcattccctcggatgaataacatgagtttttgactcctccctccctctttccttctcctactcgcctcctcaggagtagaagcaggtgctggaaccggttgaacggtgtaccctcccttagctggtaatcttgcccatgcaggagcgtctgttgatttaactattttctctcttcatttagctggtatctcatcaattctaggggcaatcaacttcattacaacagtaatcaacatgaaaccccatgccgtctctatataccaaattccactatttgtctgagccgtcctaattacagctgtccttctgctcctctcacttcctgtcttagctgccggcattacaatacttttaaccga

SEQ ID NO: 48 - DNA, 512 bp, Lichia amia
  source 1..512: mol_type = genomic DNA
agccaacctggcgctctcctaggggacgaccaaatttataacgtaatcgtaaccgcacatgctttcgtaataattttctttatagtaataccgattataatcgggggatttggaaattgattaatcccattaataattggtgcccctgacatagcatttccccgaatgaataatataagtttctggcttctccccccttctttccttcttctgcttgcttcatcaggagttgaagctggtgctggaactggttgaacagtctacccccctctatcaggaaaccttgctcacgcaggggcttctgtagacttaactattttttcacttcacctggctggaatttcttctattctcggggctattaactttattacaacagtcattaatataaaaccccatgccgtctctatgtatcaaattcctctatttgtctgagctgttcttattacagccgtccttctactactctctctgcctgttctagctgccggtattacaatactactaaccga

SEQ ID NO: 49 - DNA, 512 bp, Scomberoides commersonnianus
  source 1..512: mol_type = genomic DNA
agccaacccggggccctcctcggagacgaccaaatctataacgtcattgttacggcccatgccttcgtaataatcttctttatagtaataccaattatgatcggaggtttcggaaactgactcattcccctaataattggtgctcccgatatagctttccctcgaataaacaacataagcttctgactcctacccccttcctttcttctcctccttgcctcctcaggagttgaagctggagcaggaactggctgaacagtctaccctcccctagcaggcaacctagcccacgcaggagcatccgtagacctaaccatcttctccctccacctagccggaatttcctcaattctaggagccatcaatttcatcacaactatcattaacataaaaccccatgccgtttccatataccaaatccccctattcgtttgagccgtcctaattacagcagtccttctccttctttctttacctgttcttgccgccggcatcacaatacttctaaccga

SEQ ID NO: 50 - DNA, 26 bp, synthetic construct
  source 1..26: mol_type = other DNA
tcaaccaaccacaaagacattggcac

SEQ ID NO: 51 - DNA, 26 bp, synthetic construct
  source 1..26: mol_type = other DNA
tagacttctgggtggccaaacaatca